(19) United States
(12) Patent Application Publication
(10) Pub. No.: US 2010/0268094 A1
(43) Pub. Date: Oct. 21, 2010

(54) Consumer electronic camera photoplethysmograph

(75) Inventors: Thomas Hasling, Honolulu, HI (US); Luke Joseph, Honolulu, HI (US); Todd Blume, Honolulu, HI (US)

Correspondence Address: JAMES C. WRAY, 1493 CHAIN BRIDGE ROAD, SUITE 300, MCLEAN, VA, 22101, US

(73) Assignee: Oceanit Laboratories Inc.

(21) Appl. No.: 12/798,992

(22) Filed: Apr. 15, 2010

Related U.S. Application Data
(60) Provisional application No. 61/212,696, filed on Apr. 15, 2009.

(51) Int. Cl. A61B 5/0205

(52) U.S. Cl. 600/484

(57) ABSTRACT

A body part is placed on or near a consumer electronic device camera. The camera senses periodic light variations. A program analyses variations in images captured by the digital camera to determine pulse rate or breathing rate.

This application claims the benefit of U.S. Provisional Application No. 61/212,696, filed Apr. 15, 2009, which is hereby incorporated by reference in its entirety.

### SUMMARY OF THE INVENTION

A camera on a variety of consumer electronics (e.g. cell phones, PDAs, computers, webcams, and digital cameras) is used to detect variations in transmitted light through a finger tip or other body part, to provide photoplethysmographic data. This can be achieved using ambient light or specifically applied light (e.g. LED, lamp, display).

The technique for obtaining a pulse uses a camera equipped computing device or smart phone such as the iPhone. This invention will work equally well on handheld devices and notebook and desktop computers with built-in or attached cameras. The invention provides an application to measure one's pulse. The device's camera is used to measure the pulse, which is then displayed on the device. The invention senses pulse rate by using the camera to detect changes in transmitted light through a finger tip. The images captured by the camera are processed by the application to determine pulse rate from the variations in the images over time.

In one usage scenario, the user places their fingertip or other body part over the camera lens while running the application. The application automatically detects the presence of a pulse and displays the results, or indicates to the user that a pulse is not picked up if the image signal is insufficient to determine the pulse. Whenever pulse is detected the results are displayed. The pulse and/or pulse rate results may be displayed numerically, as a graph, as an animated graphic, or as a combination of these. The pulse and/or pulse rate results may also be indicated by audio means such as beeps, tones, or spoken words.

Another usage scenario is to trigger the application to gather image data for a period of time, and to then calculate the pulse rate in post processing of the buffered images after image capture is completed. The user is prompted when to hold their finger over the lens, and given a progress indication as the data is gathered. The user is notified when the data capture is complete, and the result is displayed.

Regardless of whether the images are captured and results displayed in a continuous mode, or the images are captured before the results are displayed in a batch mode, the fundamental innovation of the invention remains the same: analyzing variations in images captured by a digital camera sensor to detect pulses and calculate pulse rate.

It may not be necessary to place a finger tip in full coverage of the camera, if partial coverage provides the illumination variations which are indicative of pulses. Under appropriate conditions it may also be possible to detect respiration rate.

Consumer medical telemetry will find the invention useful. The closest previous products are various types of watches that capture users' pulses. There is no prior art of using an existing camera on consumer electronics to determine pulse rate. As an iPhone application in and other operating systems such as Windows Mobile software, the invention could potentially produce considerable revenue. Apple, Microsoft or other vendors associated with the mobile phone software market would use the invention. A specific application using the invention that is appealing is using the invention before, after or during exercise.

The invention provides biological rate sensing, using a consumer electronic device having a digital camera. Placing a body part near or in contact with the camera, illuminating the body part with light, and sensing short term variations in light received in the camera shows a rate of pulsed flow of blood or breathing.

Timing the short term variations in light, and averaging the timing between the short term variations provides a pulse or breathing rate. Converting the timing to variations per minute provides indications of the pulse or breathing rate per minute.

When the biological rate sensed is a pulse rate, a body part is placed on the camera.

Illuminating the body part includes using ambient light or light from a small lamp or from a display on the consumer electronic device.

The consumer electronic device has a display screen, and indicates the pulse rate on the display screen in beats per minute. The indicating includes numerically and graphically displaying the pulse rate on the display screen. The consumer electronic device has a speaker, and the indicating includes audibly vocalizing the pulse rate.

A range of acceptable pulse rates is stored. Comparing the indicated pulse rate with the stored range of acceptable pulse rates produces an audible alarm when the indicated pulse rate is out of the acceptable range. The out of range pulse rate is communicated to a remote receiver. The sensed pulse rate and time are stored.

When the body part is a chest of a user, the placing comprises placing the camera near the chest, and the indicating comprises indicating breathing rate.

The new method senses a biological rate with a cellular phone by positioning a camera on the cellular phone in contact with or near a body part and sensing periodic light variations on the camera. Providing time intervals from a clock in the cellular phone and comparing the sensed light variations and the time intervals provided by the clock indicates a biological rate with one or more numeric display, graphic display, spoken words, audible alarm, and a visual alarm and remote reporting.

The necessary parts of the new biological rate sensor include a consumer electronic device having a camera. The camera senses periodic light variations through or near a body part. A clock in the device provides time periods of the periodic light variations.

A comparator program connected to the camera and clock inputs and compares the light variations sensed by the camera with the time periods provided by the clock and provides a rate of light variations.

An indicator connected to the comparator program indicates results of a rate of light variations as a biological rate.

When a body part is positioned on or near the camera, the clock times a number of seconds, and the comparator compares the number of seconds with the number of light variations as the number or fractional number of seconds between light variations.

A converter connected to the comparator and to the indicator converts the number of the light variations and the number of seconds to cycles per minute.

The indicator has a display showing the biological rate as a numeric display and as a graphic display.

The indicator has audible sound producer that produces a synthesized voice report. Upon sounding an out of range alarm, the indicator makes a call to a remote receiver and reports a numeric or graphic display or audible sound.

One biological rate is a pulse rate. Another biological rate is a breathing rate.

These and further and other objects and features of the invention are apparent in the disclosure, which include the above and ongoing written specification, with the claims and the drawings.

### BRIEF DESCRIPTION OF THE DRAWINGS

FIG. 1 is a chart showing a photo detected pulse pattern extracted from a cell phone camera.

FIG. 2 is a schematic representation of a cell phone with a finger tip placed over a camera lens while the camera is recording a video image of light passing through the finger tip.

### DETAILED DESCRIPTION OF THE DRAWINGS

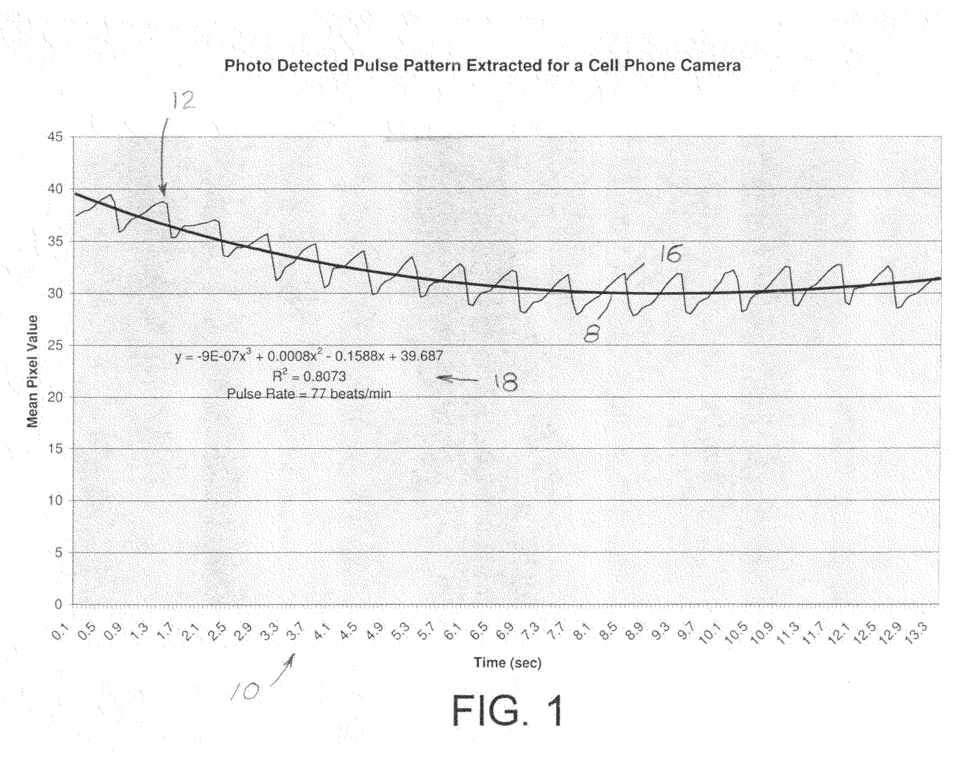

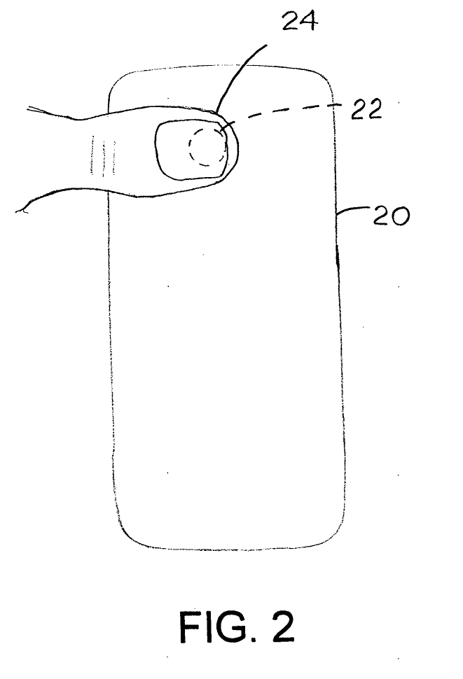

In producing the chart 10 showing a photo detected pulse pattern 12 extracted from a cell phone camera in FIG. 1, a finger tip 24 or other body part has been held over a camera lens 22 of a cell phone 20, as shown in FIG. 2. Pictures have been selected by pressing an appropriate button or touching an icon on a screen. Taking a video recording has been selected by pressing an appropriate button or touching an icon on a screen. The user has held a finger tip in place for a number of seconds while a video recording is taken by the camera in the cell phone. The seconds are counted or shown on a display. Ten seconds is usually sufficient to obtain an accurate pulse reading. Then a button or icon is pressed or touched to end the video session.

Shortly or immediately thereafter the screen displays the number of pulses per minute. The formula 18 used in the computations is shown in FIG. 1 and is programmed into the phone.

y=−9E−07x³+0.0008x²−0.1588x+39.687

R²=0.8073

Pulse Rate=77 beats/min

If undue absence, unevenness or excessiveness of pulse rate is noted during the video recording, an alert may sound, the screen may flash or the cell phone may dial a pre-inserted number for a remote alert.

When placing a finger tip on the camera lens, an often adjacent light sensor may be covered with the finger, or care may be taken not to cover the light sensor to improve accuracy of the pulse sensing by light variations sensed through the finger tip on the lens.

Instead of a finger tip, another part of the body may be placed against the lens. For example, the lens may be positioned in front of or behind an ear lobe while the source of light illuminates the other side of an ear. An arterial bump on an inside of a wrist may be held lightly against the camera lens so that light repeatedly passes through or around the arterial bump as blood pulses through.

The changes 16 in transmissivity, which is video recorded through the camera lens, and is measured as lumens on the lens may be in the order of about 10%, such as shown in FIG. 1. The changes may be larger or smaller. The average pixel illumination 8 is shown in FIG. 1, but may not be necessary to be shown or determined to record the periodic changes 16 in transmissivity which are indicative of pulses.

The change 16 in the average pixel illumination 8 shown in FIG. 1 shows that the changes due to pulse rate are recorded in varied illumination.

It may not be necessary to place a finger tip in full coverage of the camera, if partial coverage provides the illumination variations which are indicative of pulses.

Although this example describes the use of a video camera to extract data, the device's camera need not be specifically a video camera. The only requirement is that the application can acquire image data from the camera at a sufficient rate to detect pulses. Time is therefore not a constraint in this invention and the data can be post processed or processed real time. This in effect allows the detection of a pulse using camera phones that do not have full video recording capability.

While the invention has been described with reference to specific embodiments, modifications and variations of the invention may be constructed without departing from the scope of the invention, which is defined in the following claims.

We claim:

1. A method comprising:
biological rate sensing,
using a consumer electronic device having a digital camera,
placing a body part near or in contact with the camera,
illuminating the body part with light,
sensing short term variations in light received in the camera,
timing the short term variations in light,
averaging the timing between the short term variations,
converting the timing to variations per minute,
indicating the rate of variations per minute.

2. The method of claim 1, wherein
the biological rate sensing is pulse rate,
the placing comprises placing a body part on the camera, and
the indicating comprises indicating the pulse rate.

3. The method of claim 2, wherein the illuminating the body part comprises illuminating the body part with ambient light.

4. The method of claim 2, wherein
the consumer electronic device has a display screen, and
the indicating comprises indicating the pulse rate on the display screen in beats per minute.

5. The method of claim 4, wherein the indicating comprises numerically and graphically displaying the pulse rate on the display screen.

6. The method of claim 2, wherein
the consumer electronic device has a speaker, and
the indicating comprises audibly vocalizing the pulse rate.

7. The method of claim 6, further comprising
storing a range of acceptable pulse rates,
comparing the indicated pulse rate with the stored range of acceptable pulse rates producing an audible alarm when the indicated pulse rate is out of the range.

8. The method of claim 7, further comprising
communicating the out of range pulse rate with a remote receiver.

9. The method of claim 2, further comprising storing the sensed pulse rate and time.

10. The method of claim 1, wherein the body part is a chest of a user, the placing comprises placing the camera near the chest, and the indicating comprises indicating breathing rate.

11. A method comprising sensing a biological rate with a cellular phone, positioning a camera on the cellular phone in contact with or near a body part and sensing light variations on the camera, providing time intervals from a clock in the cellular phone, comparing the sensed light variations and the time intervals provided by the clock in the cellular phone and indicating a biological rate with one or more numeric display, graphic display, spoken words, audible alarm, visual alarm and remote reporting.

12. Biological rate sensor apparatus comprising:
a consumer electronic device having
a camera sensing periodic light variations through or near a body part,
a clock providing time periods,
a comparator connected to the camera and clock comparing the light variations sensed by the camera with the time periods provided by the clock and providing a rate of light variations,
an indicator connected to the comparator indicating results of the providing a rate of light variations as a biological rate.

13. The apparatus of claim 12, wherein a body part is positioned on or near the camera, the clock times a number of seconds, the comparator compares the number of seconds with the number of light variations as the number or fractional number of seconds between light variations.

14. The apparatus of claim 12, further comprising a converter connected to the comparator and to the indicator for converting the number of the light variations and the number of seconds to variations per minute.

15. The apparatus of claim 12, wherein the indicator further comprises a display showing the biological rate as a numeric display and as a graphic display.

16. The apparatus of claim 12, wherein the indicator comprises an audible sound producer produces a synthesized voice report and an out of range alarm.

17. The apparatus of claim 12, wherein the indicator comprises a remote reporter of a numeric or graphic display or audible sound to a remote receiver.

18. The apparatus of claim 12, wherein the biological rate is a pulse rate.

19. The apparatus of claim 12, wherein the biological rate is a breathing rate.
